(19) United States
(12) Patent Application Publication
(10) Pub. No.: US 2004/0077864 A1
(43) Pub. Date: Apr. 22, 2004

(54) Method for preparing chiral amines

(76) Inventors: Mahn-Joo Kim, Pohang-city (KR); Yangsoo Ahn, Seoul (KR); Yoon Kyung Choi, Kyungju-city (KR); Mi Jung Kim, Bucheon-city (KR)

Correspondence Address: BAKER & BOTTS, 30 ROCKEFELLER PLAZA, NEW YORK, NY, 10112

(21) Appl. No.: 10/467,122

(22) PCT Filed: Dec. 6, 2002
(86) PCT No.: PCT/KR02/02297

(30) Foreign Application Priority Data
Dec. 6, 2001 (KR) 10-2001-0077030

(51) Int. Cl. C07D 471/02; C07D 491/02; C07D 498/02; C07D 333/12; C07D 213/26

(52) U.S. Cl. 546/114; 546/329; 549/74; 549/491; 546/115

(57) ABSTRACT

Disclosed is a method of preparing chiral amine. The method includes reacting ketoxime, palladium, lipase, acyl-donating compound, and tertiary amine to prepare amide, and amide is hydrolyzed.

### BACKGROUND OF THE INVENTION

1. Field of the Invention

The present invention relates to a method of preparing chiral amines, and more preferably, to a method of preparing chiral amines by simple procedures using starting materials which are easy to handle.

2. Background of the Invention

The procedures for preparing chiral amines are classified into two categories: chemical procedures using metal catalysts and biochemical procedures using an enzyme catalyst. The chemical procedure and the biochemical procedures have complementary advantages and shortcomings. Thus, the combination of the two catalysts has been attempted the preparation of chiral amines. Till now, only one method reported by a German group (Reetz, M. T; Schimossek, K. Chimia, 1996, 50. 668) utilized the enzyme-metal combination for preparing chiral amines.

In this method, a chiral amine was prepared as optically pure amide by dynamic kinetic resolution from the mixture of racemic 1-phenylethylamine as a substrate, palladium as a racemization catalyst, and lipase as a selective acylation catalyst. The optically pure amide is formed by selective acylating the desired enantiomer with an acylating agent in the presence of lipase while the other enantiomer is simultaneously racemized in situ by the action of the palladium catalyst. The reaction was performed at a temperature of 50 to 55° C. for 9 days, and the conversion was 75 to 77%.

However, the method suffers from that it was applicable to only on substrate and required a long reaction time and for a modest yield.

### SUMMARY OF THE INVENTION

It is an object of the present Invention to provide a method for preparing chiral amines with high yields and excellent optical purities within a shorter reaction time from ketoxime which is readily synthesized from ketone, by the combination of a metal catalyst and a biocatalyst.

These and other objects may be achieved by a method for preparing chiral amines by reacting ketoxime represented by formula I, palladium, lipase, an acyl donor, and a tertiary amine in an organic solvent to prepare an amide represented by formula IV, and then hydrolyzing the amide.

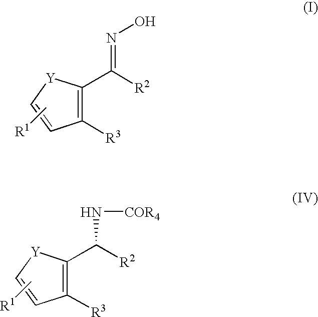

(wherein

R¹ is hydrogen, an alkyl, an alkoxy, phenyl, or a phenyl substituted with an alkyl;

R² and R³ are each independently, hydrogen or and an alkyl, or R² and R³ bond together to form a ring, where the alkyl is C₁₋₃ alkyl substituted with hydrogen, oxygen, nitrogen, sulfur, or a halogen, and the ring is represented by —(CH₂)ₙ—X—, where n is an integer between 1 to 3;

X is methylene, oxygen, sulfur or nitrogen;

Y is —CH═CH—, —CH═N—, sulfur or oxygen; and

R⁴ is C₁₋₅ alkyl substituted with oxygen or a halogen.)

### DETAILED DESCRIPTION OF THE INVENTION

The present invention relates to a method for preparing chiral amines, which may be useful as an intermediate in the production of medicines from ketoximes, which are easy to make and handle.

In the present invention, ketoxime represented by formula I, palladium as a reduction and racemization catalyst, a lipase as a stereo selective acylation catalyst, an acyl donor, and a tertiary amine react in an organic solvent to provide a chiral amide represented by formula IV.

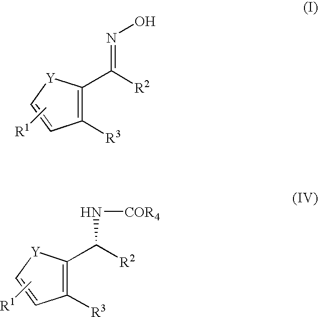

(wherein R¹, R², R³, Y, and R⁴ are defined as above)

In detail procedure, the palladium catalyst is activated in the presence of hydrogen gas at a temperature between 40 to 100° C. for 30 minutes to 1 hour. The activated catalyst is then cooled to room temperature, and ketoxime represented by formula I as a substrate, a lipase as an acylation catalyst, an acyl donor, a tertiary amine, and an organic solvent are added. The reaction bath is charged with 1 atm of hydrogen gas. The reaction mixture is preferably performed at a temperature between 40 and 70° C.

The palladium catalyst may be palladium powder, palladium black, or palladium (valence number: 0), supported on carbon, barium sulfate, barium carbonate, or calcium carbonate, and preferably palladium supported on carbon, barium sulfate, barium carbonate or calcium carbonate.

The commercially available supported palladium includes 5 to 10% of palladium. In case that the supported palladium has a palladium content of 5%, the amount of palladium catalyst is preferably 40 to 70% based on the weight of the ketoxime.

The formulas IIR and IIS represent the enantiomers of racemic amine formed by the reaction.

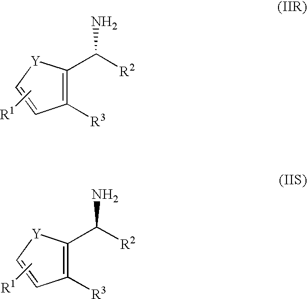

(wherein R¹, R², and R³ are defined as above.)

The lipase catalyzes selective acylation of the enantiomer represented by formula IIR in the presence of the acyl donor to produce the optically pure amide represented by formula IV.

The other enantiomer, represented by formula IIS is racemized in situ by the tertiary amine and palladium to form the compound of formula IIR. The compound of IIR is continuously converted into an amide represented by formula IV by the enzymatic acylation reaction.

Examples of lipase are Pseudomonas ceoacia lipase (e.g. lipase PS-C immobilized on ceramic, or lipase PS-D immobilized on diatomite (Japan, Amano-Enzymes Inc.), and Candida antarctica lipase (e.g. immobilized on acrylic resin, Novozym 435, Nove Nordisk Korea) are preferable.

The amount of the immobilized lipase is preferably 1 to 3 times that of the weight of ketoxime based on weight.

The acyl donor is represented by formula III, and the examples thereof are ethyl acetate, 2,2,2-trifluoroethyl acetate, 2,2,2-trichloroethyl acetate, and p-chlorophenyl acetate. The amount of the acyl donor is preferably 1.5 to 2 equivalents based on 1 equivalent of ketoxime.

R⁴CO₂R⁵ (III)

(wherein

R⁴ is defined as above; and

R⁵ is hydrogen, C₁₋₃ alkyl substituted with a halogen, oxygen, nitrogen or sulfur, C₁₋₃ alkenyl, phenyl or phenyl substituted with a halogen)

The tertiary amine is represented by formula V, and the examples thereof are triethylamine and diisopropylethylamine. The amount of the tertiary amine is 1 to 5 equivalents based on 1 equivalent of ketoxime.

R⁶₃N (V)

(wherein R⁶ is a C₁₋₃ alkyl)

The organic solvent may be benzene, toluene, xylene, tetrahydrofuran, dioxane, methylenechloride, or t-butyl methyl ether. The amount of the organic solvent is preferably controlled between 0.05 to 0.25M based on the concentration of ketoxime used.

After the complete reaction, the palladium catalyst and lipase are filtered off, and the optically pure amide was separated by column chromatography.

The amide is hydrolyzed to provide optically pure amine that is useful as an intermediate. The hydrolysis is well known In the related art, so a detailed description thereof will be omitted.

The method for preparing a chiral amine according to the present invention is shown in scheme I.

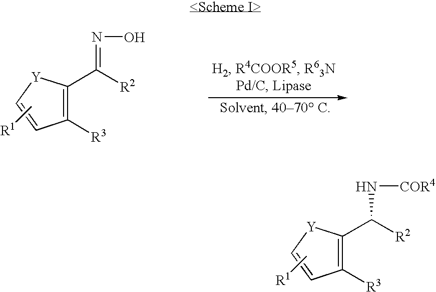

The present invention is further explained in more detail with reference to the following examples, but the examples should not be construed as limiting the scope of the claimed invention.

### EXAMPLE 1

Palladium on activated carbon (content of palladium: 5%, 34 mg) was activated in the presence of hydrogen gas at a temperature of 40° C. for 30 minutes. Acetophenone hydroxime (50 mg, 0.37 mmol) and 100 mg of novozym 435 (Nove Nordisk Korea) and 3.6 ml of toluene were introduced under an argon atmosphere into the reaction vessel in which activated palladium on activated carbon (content of palladium: 5%, 34 mg) was placed.

To the resulting mixture, ethyl acetate (72.3 μl, 0.74 mmol) and dilsopropylethylamine (193 μl, 1.11 mmol) were added, and deoxygenation occurred under vacuum. The reaction vessel was charged with 1 atm. of hydrogen gas and stirred at 60° C. for 5 days.

After the complete reaction, the reaction mixture was filtered and subjected to column chromatography to provide (R)-N-acetyl-1-phenylethylamine. The isolated product was dissolved into 1.2N HCl solution, then refluxed for 9 hours, cooled, and neutralized to obtain a desired amine.

The final chemical structure of chiral amine derivative was identified by ¹H NMR and ¹³C-NMR, and the optical purity which were determined with a chiral high-performance liquid chromatography (equipped with Whelk-01 or Chiraldex OD-H column), was 95% ee, and the yield was 80%.

### EXAMPLES 2 to 8

Optically pure amines were prepared by the same procedure as in Example 1, except that oxime as shown in Table 1 was used instead of acetophenone hydroxime.

The yields and optical purities of the chiral amines according to Examples 1 to 8 are shown in Table 1.

TABLE 1

| | | Con- | | Optical |
|---|---|---|---|---|
| | Substrate | version | Yield | purity |
| Example 1 | 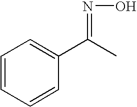 | >68% | 80% | 98% |
| Example 2 | 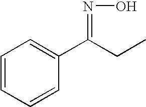 | >98% | 76% | 98% |
| Example 3 | 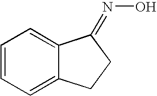 | >98% | 84% | 95% |
| Example 4 | 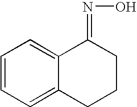 | >98% | 70% | 97% |
| Example 5 | 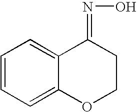 | >98% | 89% | 99% |
| Example 6 | 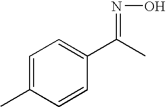 | >98% | 84% | 97% |
| Example 7 | 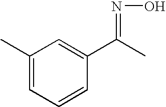 | >98% | 81% | 94% |
| Example 8 | 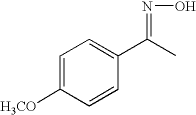 | >98% | 82% | 96% |

It is evident from Table 1 that the optically pure amines are prepared with high optical purity (94-99% ee) and high yield (70-89%) from the ketoximes using the combination of the palladium catalyst which catalyzes both reduction of ketoxime and the racemization of the resulting amines, and a lipase which catalyzes enantioselectively the acylation of amine. These results Indicate that the present invention provides the methods for the efficient preparation of chiral amines.

The method of the present invention provides the preparation of chiral amines in the form of an amide from achiral ketoximes by the combination of a palladium and a lipase and has advantages that it uses readily available ketoximes as the substrates and provides high yields and excellent enantiopurities.

Since it is applicable for preparing various amines, and the method provides a useful alternative for the conventional chemical or biochemical procedures. The chiral amines prepared by the method of the present invention can be used as chiral building blocks for the synthesis of medicines or fine chemicals.

What is claimed is:

1. A method for preparing chiral amine, comprising:
reacting ketoxime represented by formula 1, a palladium catalyst, a lipase, an acyl donor, and a tertiary amine in an organic solvent to prepare an amide of formula IV; and
hydrolyzing the amide. 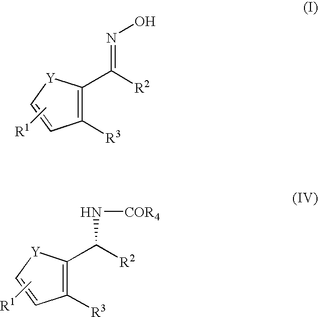
(wherein
R¹ is hydrogen, alkyl, alkoxy, phenyl, or phenyl substituted with alkyl;
R² and R³ are the same or independently hydrogen or alkyl, or R² and R³ bond together to form a ring, where the alkyl is C₁₋₃ alkyl substituted with hydrogen, oxygen, nitrogen, sulfur, or a halogen, and the ring is represented by —(CH₂)ₙ—X—, n being an integer between 1 to 3;
X is methylene, oxygen, sulfur or nitrogen;
Y is —CH═CH—, —CH═N—, sulfur or oxygen; and
R⁴ is a C₁₋₅ alkyl substituted with oxygen or a halogen.)

2. The method of claim 1, wherein the palladium catalyst is selected from the group consisting of palladium powder; palladium black; and palladium supported on carbon, barium sulfate, barium carbonate, or calcium carbonate.

3. The method of claim 1, wherein the amount of the palladium catalyst is 40 to 70% based on the weight of the ketoxime.

4. The method of claim 1, wherein the lipase is immobilized Pseudomonas cepacia lipase or immobilized Candida antarctica lipase.

5. The method of claim 1, wherein the amount of the lipase is 1 to 3 times based on the weight of the ketoxime.

6. The method of claim 1, wherein the acyl donor is represented by formula III:
R⁴CO₂R⁵ (III)
(wherein
R⁴ is a C₁₋₅ alkyl substituted with a halogen or oxygen;
R⁵ is a C₁₋₃ alkyl substituted with hydrogen, oxygen, nitrogen, sulfur, or halogen, or C₁₋₃ alkenyl, phenyl, or phenyl substituted with a halogen.)

7. The method of claim 1, wherein the amount of the acyl donor is 1.5 to 2 equivalents based on 1 equivalent of the ketoxime.

8. The method of claim 1, wherein the tertiary amine is represented by formula V:
R⁶₃N
(wherein, R⁶ is C₁₋₃ alkyl.)

9. The method of claim 1, wherein the amount of the tertiary amine is 1 to 3 equivalents based on 1 equivalent of the ketoxime.

10. The method of claim 1, wherein the reaction is performed at 40 to 70° C.

11. The method of claim 1, wherein the amount of the organic solvent is controlled between 0.05 to 0.25 M based on the concentration of ketoxime.
